(19) United States
(12) Patent Application Publication
(10) Pub. No.: US 2013/0092163 A1
(43) Pub. Date: Apr. 18, 2013

(54) Stay put cannula

(71) Applicant: Oreste Louis Puccini, SR., Albuquerque, NM (US)

(72) Inventor: Oreste Louis Puccini, SR., Albuquerque, NM (US)

(21) Appl. No.: 13/317,193

(22) Filed: Oct. 13, 2011

(51) Int. Cl. A61M 16/00

(52) U.S. Cl. 128/204.18

(57) ABSTRACT

The Stay Put Cannula is a medical device which will be used to place and keep gas (usually oxygen) delivery tubes in the patient's nostrils so that the nostril tubes do not easily fall out or are moved. The Stay Put Cannula can also be used to keep other similar tubes such as intravenous tubes in place as well. The Stay Put Cannula can also save nurses and other medical providers time and effort by not having to frequently check and reinsert the nostril tube.

### BACKGROUND OF THE INVENTION

1. Field of the Invention

The Stay Put Cannula invention is a medical device to hold oxygen tubes on a patients' face so that the venting holes remain in the patient's nostrils and not slip out as frequently occurs in elderly or comatose patients.

2. Description of Prior Art

The “Cannula” has been in existence for a long time. The Cannula tube is the source of a gas, usually oxygen, needed for medical purposes to the patient. It is usually a soft pliabel tube that is placed around the patients' ears and the two exhaust holes are short extensions, usually about ¼″ long, that are placed in the patient's nostrils. However, due to the tubes flexibility and patient movement, these extensions frequently fall out of the nostrils releasing the oxygen into the air without benefitting the patient.

The applicant, customer, is unaware of any prior patent or history of the original “cannula.” Applicant has never see a cannula similar to the Stay Put Cannula.

### SUMMARY OF THE INVENTION

The present invention is an improvement on the cannula to hold the cannula in place to maximize the benefit to the patient.

The applicant is Oreste L. Puccini, Sr., and I invented the “Stay Put Cannula” on Aug. 3, 2005, as I noticed my bedridden wife's, now deceased, oxygen delivery tubes frequently were out of her nostrils and entirely off her face for long periods of time. The original medical name for this oxygen delivery mask is CANNULA.

1. The STAY PUT CANNULA consists of a clear plastic hose or tube about seven feet long delivering oxygen to a patient from a compressed oxygen tank or from an oxygen machine. A 44 inch plastic loop is attached o this tube dividing the oxygen gas into two smaller diameter tubes making the face mask.
2. A plastic restraint holds these two smaller tubes into the patient's nostrils, which together help holding the tubes in place and over the patient's ears.
3. One flexible stainless steel wire about 44 inches long and of small gage is to be inserted into the plastic tube on the face mask. Fitting the wire into the plastic tube can be done at any one of th joints of the face mask.
4. It is important to insure that the stainless steel wire be heated on both ends of the wire to form beads. The beads will insure that the wire does not puncture the hose. This wire in the tube would be bent (hand pressure) to fit over patients ears and hold the Stay Put Cannula in place, much the same as a pair of eye glasses!
5. The Stay Put Cannula plastic face hoses or tube may require a larger diameter as the stainless steel wire may restrict oxygen air flow or a smaller gage wire may ease oxygen air flow.

### BRIEF DESCRIPTION OF THE DRAWINGS

FIG. 1 is a drawing of the Stay Put Cannula. A Flexible Stainless Steel Wire (1) is inserted inside the Flexible Plastic Tubes (2) with the Nostril Tube (3) marked. A Bead (4) is secured on each end of the wire. The Holding Bracket (5) is inserted over the outside of the tubes and the tubes are inserted back into the Connector (6) that is secured to the Gas Source Tube (7).

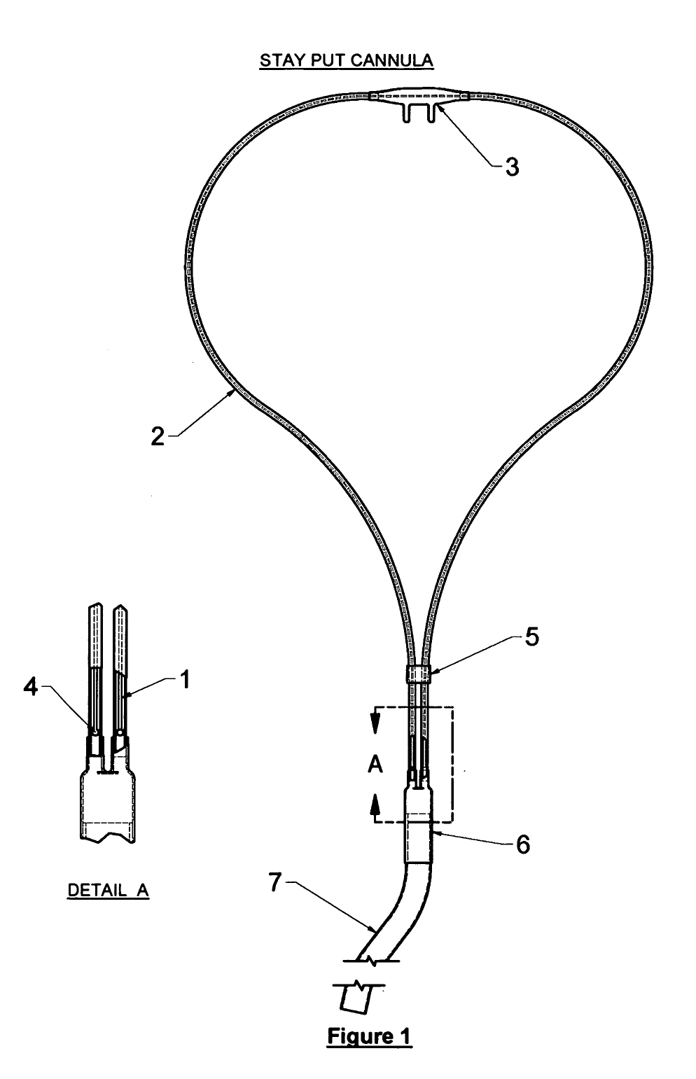

### DETAILED DESCRIPTION OF INVENTION

The present invention is an improvement on the cannula to hold the cannula in place to maximize the benefit to the patient.

The claim

1

is:

1. The Stay Put Cannula is a simple device to hold the gas (oxygen) delivery tubes in place by means of a flexible stainless steel wire or similar rust resistant wire or material with sufficient rigidity to hold the tube in place.
The claim 2 is:

2. Claim number 2 is an improvement or modification on the Cannula by more effectively delivering the oxygen to patients especially the many patients who are unaware that the nostril tubes have fallen out and this kind of patient frequently does not replace the nostril tubes.
